(19) United States
(12) Patent Application Publication
(10) Pub. No.: US 2020/0060736 A1
(43) Pub. Date: Feb. 27, 2020

(54) PLANTARLY APPLIED REVERSE LOCKING PLATE

(71) Applicants: James Connors, Broadview Heights, OH (US); Craig Verdin, North Royalton, OH (US); Allan M. Boike, Hinckley, OH (US); Nilin Rao, Denver, CO (US)

(72) Inventors: James Connors, Broadview Heights, OH (US); Craig Verdin, North Royalton, OH (US); Allan M. Boike, Hinckley, OH (US); Nilin Rao, Denver, CO (US)

(73) Assignee: Kent State University, Kent, OH (US)

(21) Appl. No.: 16/546,735

(22) Filed: Aug. 21, 2019

Related U.S. Application Data
(60) Provisional application No. 62/720,373, filed on Aug. 21, 2018.

(51) Int. Cl. A61B 17/72

(52) U.S. Cl. CPC A61B 17/7291; A61B 2017/681

(57) ABSTRACT

The present invention provides a method of placing a plate fixation device in a foot comprising the steps of: plantarly placing a reverse locking plate on the foot; and utilizing a locking guide to dorsally place reverse locking screws into the foot to mate with the reverse locking plate.

### CROSS-REFERENCE TO RELATED APPLICATION

This application claims priority to U.S. Provisional Application No. 62/720,373 entitled “Plantarly Applied Reverse Locking Plate” and filed on Aug. 21, 2018, which is herein incorporated by reference.

### FIELD OF THE INVENTION

The present invention relates to a method and device for use in fixation of the metatarsal bones of the foot for fracture reduction, hallux valgus correction, medial column tarsal/metatarsal fusions and similar surgical procedures. The present invention further relates to the use of a plantarly applied reverse locking plate to perform these surgeries. The present invention furthermore relates to the use of reverse locking screws and a targeting guide in order to place the plantarly applied reverse locking plate with minimal dissection.

### BACKGROUND OF THE INVENTION

Bunions have long been one of the more common types of painful foot deformities. The technical name for this type of deformity is Hallux Abducto Valgus (HAV), which is generally described as a medial deviation of the first metatarsal accompanied by a lateral deviation and/or valgus rotation of the hallux (or “big toe”). This condition can lead to painful motion of the first metatarsophalangeal (MTP) joint, and adjustments in gait that may ultimately cause problems further up the leg.

Historically, many different surgical procedures have been proposed to correct HAV deformities. The goals of surgical approaches to the treatment of a hallux valgus deformity are to relieve symptoms and pain, restore function and correct or reduce the deformity. For some indications, the surgical treatment is limited to removal of a medial portion of the metatarsus to reduce the prominence of the bunion. In more severe cases, or for elderly patients, resectional arthroplasty of the metatarso-phalangeal (MTP) joint, with or without implant, or joint arthrodesis may be indicated, although these procedures result in loss of motion at that joint.

However, in the majority of cases, the surgical treatment involves a osteotomy to correct structural deformities associated with the first metatarsal bone and the hallux phalanges, relieve symptoms and pain and restore proper foot function. The osteotomy results in the creation and correction of metatarsal segments used to return the metatarsus and MTP joint to their normal anatomical positions and restore acceptable hallux valgus. In any osteotomy, it is essential that a fixation device, such as screws, K-wires, pins, plates and the like, be used to maintain correction of the metatarsal segments until bone union occurs.

One well-known surgical operation used for correcting HAV deformities is the Lapidus-type procedure. As first introduced in 1934, the Lapidus procedure involves completing an arthrodesis or fusion of the first metatarsocuneiform joint (MCJ) in the foot in order to attain a desired HAV correction. The MCJ consists of the first metatarsal long bone of the foot and the first cuneiform bone of the foot which is located just proximal or behind the first metatarsal bone. An arthrodesis or fusion consists of connecting bones together to relieve pain associated with the two bones of the MCJ rubbing together and causing pain. Arthrodesis, also known as artificial ankylosis or syndesis, is the artificial induction of joint ossification between two bones via surgery. This is done to relieve intractable pain in a joint which cannot be managed by pain medication, splints or other normally indicated treatments.

The object of the Lapidus procedure is to realign the two joint surfaces between the first metatarsal bone and the first cuneiform bone in order to achieve correction of the HAV deformity. Various methods of temporary correction are utilized whether manually, or with the use of instruments or guides to temporarily achieve the appropriate correction position of the MCJ to achieve correction of the HAV deformity.

Once the Lapidus procedure has been performed and the MCJ is positioned properly to achieve correction of the HAV deformity, the MCJ has been previously fixated with various types of screws, metal K-wires, dorsal plates, medial plates, and offset medial plates, but most notably with either screws or medial plates. The fixation techniques reported vary in steps, screw diameter size used, K-wires used, placement, plates, etc.

Historically, the Lapidus procedure has encountered various reported issues with achieving the object of fusing or obtaining an arthrodesis of the MCJ. There are reported problems with non-unions occurring and requirements for the patient to be non-weight bearing for significant periods in order to achieve the union or arthrodesis of the MCJ. A non-union occurs when the two bones of the MCJ fail to fuse together after the Lapidus procedure. Therefore, there is a need in the art for a method and device for performing the Lapidus procedure that minimizes these problems and associated risks.

### SUMMARY OF THE INVENTION

In a first embodiment, the present invention provides a method of placing a plate fixation device in a foot comprising the steps of: plantarly placing a reverse locking plate on the foot; and utilizing a locking guide to dorsally place reverse locking screws into the foot to mate with the reverse locking plate.

In a second embodiment, the present invention provides a method as in any embodiment above, wherein the reverse locking plate includes a plurality of fixation apertures, a plurality of locking apertures, and a plurality of locking pin-holes located adjacent each of the plurality of locking apertures.

In a third embodiment, the present invention provides a method as in any embodiment above, wherein the locking guide includes a locking piece comprising an aperture and two locking pins.

In a fourth embodiment, the present invention provides a method as in any embodiment above, further comprising the step of temporarily fixating the locking plate at a medial aspect of the foot by utilizing the plurality of fixation apertures.

In a fifth embodiment, the present invention provides a method as in any embodiment above, further comprising the step of plantarly placing the locking guide by having the locking pins lock into place with locking pin-holes such that the locking guide aperture is aligned with one of the plurality of locking apertures of the reverse locking plate.

In a sixth embodiment, the present invention provides a reverse locking plate for use in foot surgery comprising: a body having an upper and lower surface with a side portion located between said upper and lower surface; a plurality of fixation apertures protruding from the side portion to lie above said upper surface; a plurality of locking apertures running from the upper to lower surface of the body; and a plurality of locking pin-holes located adjacent each of the plurality of locking apertures on the lower surface of the body.

In a seventh embodiment, the present invention provides reverse locking plate made from a metal selected from the group consisting of medical grade surgical steel or titanium.

In an eight embodiment, the present invention provides a kit for use in foot surgery, wherein the kit comprises a reverse locking plate and a locking guide.

In a ninth embodiment, the present invention provides a kit wherein the reverse locking plate comprises a body having an upper and lower surface with a side portion located between said upper and lower surface; a plurality of fixation apertures protruding from the side portion to lie above said upper surface; a plurality of locking apertures running from the upper to lower surface of the body; and a plurality of locking pin-holes located adjacent each of the plurality of locking apertures on the lower surface of the body.

In a tenth embodiment, the present invention provides a kit wherein the locking guide comprises a vertical arm, a lower set arm, and an upper adjustable arm.

In an eleventh embodiment, the present invention provides a kit wherein the lower set arm includes a locking piece at a distal end of the lower set arm wherein the locking piece includes an aperture and two locking pins.

In a twelfth embodiment, the present invention provides a kit wherein the upper adjustable arm is adjustable along the vertical arm and is adjusted by use of an adjustment means and wherein the upper adjustable arm includes an aperture at a distal end of the upper adjustable arm.

### BRIEF DESCRIPTION OF THE DRAWINGS

The features and advantages of the example embodiments described herein will become apparent to those skilled in the art to which this disclosure relates upon reading the following description, with reference to the accompanying drawings, in which:

FIG. 1A illustrates a top view of a human foot experiencing the condition of HAV;

FIG. 1B illustrates a top view of a human foot with one embodiment of the reverse locking plate of the present invention having been plantary placed between the medial cuneiform and first metatarsal;

FIG. 2A illustrates a top view of one embodiment of the reverse locking plate of the present invention;

FIG. 2B illustrates a perspective view of the reverse locking plate of FIG. 2A;

FIG. 2C illustrates a bottom view of one embodiment of the reverse locking plate of the present invention;

FIG. 3A illustrates a perspective view of a targeting guide of the present invention utilized in the placing of the reverse locking plate of the present invention;

FIG. 3B illustrates a side perspective view of the locking piece of the targeting guide of FIG. 3A;

FIG. 4 illustrates a perspective view of both a cannulated screw and solid screw being screwed into the reverse locking guide of the present invention;

FIG. 5 illustrates a side perspective view of a foot having the reverse locking plate of the present invention being placed; and

FIG. 6 illustrates a perspective view of a cannulated measuring guide of the present invention utilized in the placing of the reverse locking plate of the present invention.

### DETAILED DESCRIPTION OF ILLUSTRATIVE EMBODIMENTS

Disclosed are one or more devices and methods for use in fixation of the metatarsal bones of the foot for fracture reduction, hallux valgus correction, and similar surgical procedures. Similar surgical procedures include but are not limited to navicular cuneiform, talonavicular, first metatarsophalangeal or expanded for complete medial column joint (first tarsometatarsal, navicular cuneiform and talonavicular joint) fusions. The purpose of these devices and methods of their use is to allow for a surgeon to make a more familiar medial dorsal midfoot incision over the first tarsometatarsal joint when performing such surgeries as listed above.

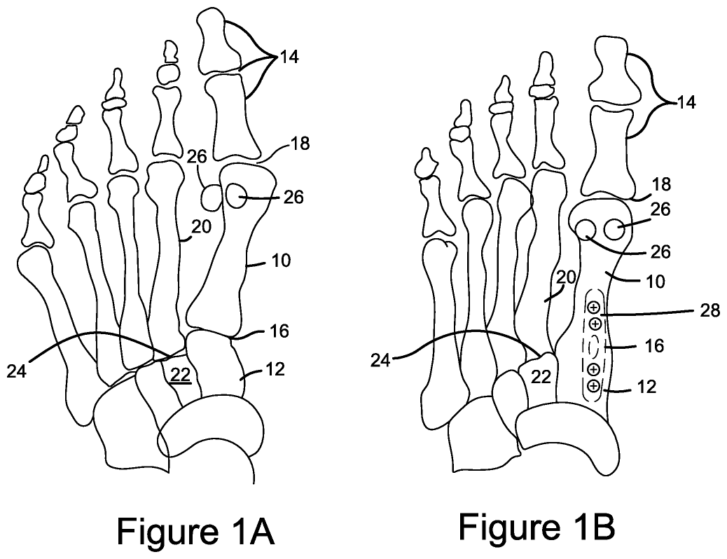

FIG. 1A illustrates the resulting anatomical configuration of a human foot experiencing HAV. In a normal human foot, the first metatarsal 10 extends between the medial cuneiform 12 and the hallux 14. The first metatarsal 10 articulates with the medial cuneiform 12 at the first metatarsocuneiform joint 16 at its most proximal aspect; and is further connected to the hallux 14 at the metatarsophalangeal joint 18 at its most distal aspect. Located adjacent to the first metatarsal 10 is the second metatarsal 20, which articulates with the intermediate cuneiform 22 at the second metatarsocuneiform joint 24. The sesamoids 26 are located beneath (plantar to) the first metatarsal head at the first metatarsocuneiform joint 16, and articulates with the head of the first metatarsal 10.

However, in a human foot experiencing HAV, such as in FIG. 1A, the first metatarsal 10 extends from the medial cuneiform 12 and deviates medially while the hallux 14 deviates laterally. As a result, the sesamoids 26 may rotate with the first metatarsal 10. This condition may lead to painful motion of the big toe joint and/or difficulty fitting footwear. Other conditions associated with HAV may include: hammer toe formation of the adjacent toes, forefoot pain on the ball of the foot (aka metatarsalgia), stress fractures of the adjacent metatarsals, flat feet (pes planus), and arthritis of the first MTPJ or midfoot. As will be explained below in greater detail, with the use of locking plate 28 of the present invention, the foot will return to its normal anatomical configuration, such as shown in FIG. 1B.

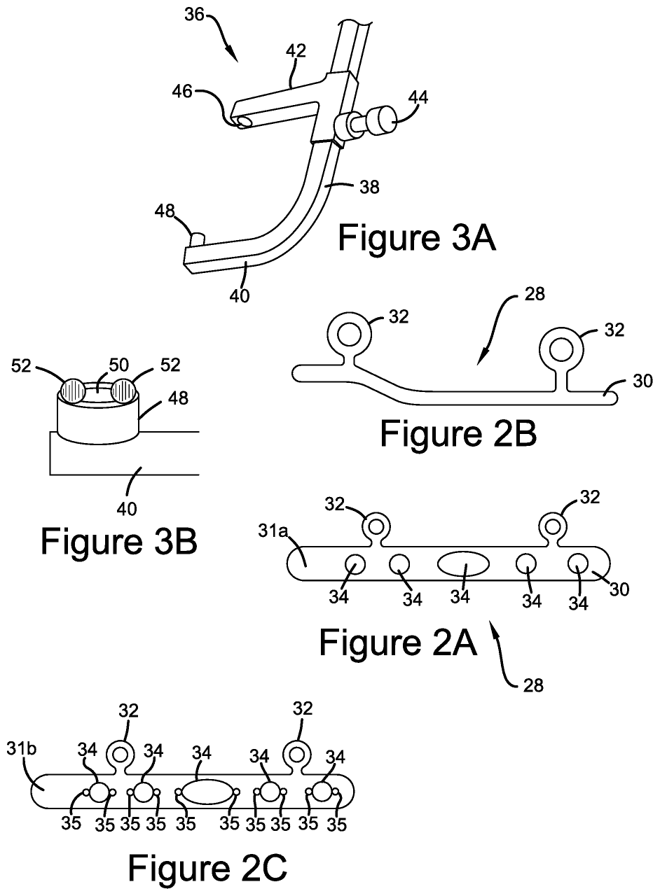

FIGS. 2A and 2B show a plantarly applied reverse locking plate 28 of the present invention. In one embodiment of the present invention, locking plate 28 comprises a body 30 with a top 31a and bottom 31b, with two fixation apertures 32 protruding from a side of top 31a and five locking apertures 34. In other embodiments there could be more than two fixation apertures 32. In yet other embodiments there could be more or less than five locking apertures 34. FIG. 2C shows bottom 31b of locking plate 28. Adjacent both sides of each locking aperture 34 are locking pin-holes 35, whose use will be explained in more detail below. In one or more embodiments of the present invention, locking plate 28 is made of a metal selected from the group consisting of medical grade surgical steel or titanium. In other embodiments of the present invention, locking plate 28 can be manufactured by additive manufacturing, such as 3D printing utilizing materials selected from the group consisting of medical grade surgical steel or titanium.

FIG. 3A of the present invention shows locking guide 36 of the present invention. Locking guide 36 comprises a vertical arm 38, a lower set arm 40, and a upper adjustable arm 42. The position of upper adjustable arm 42 can be adjusted in a vertical direction along vertical arm 38 by use of an adjustment means 44. Upper adjustable arm 42 is adjustable along vertical arm 38 so as to accommodate different sized feet needing to have locking plate 28 placed. Upper adjustable arm 42 also contains an aperture 46 at its distal end and lower set arm 40 contains a locking piece 48 at its distal end. Locking piece 48 is shown in greater detail at FIG. 3B. Locking piece 48 comprises an aperture 50 and two locking pins 52.

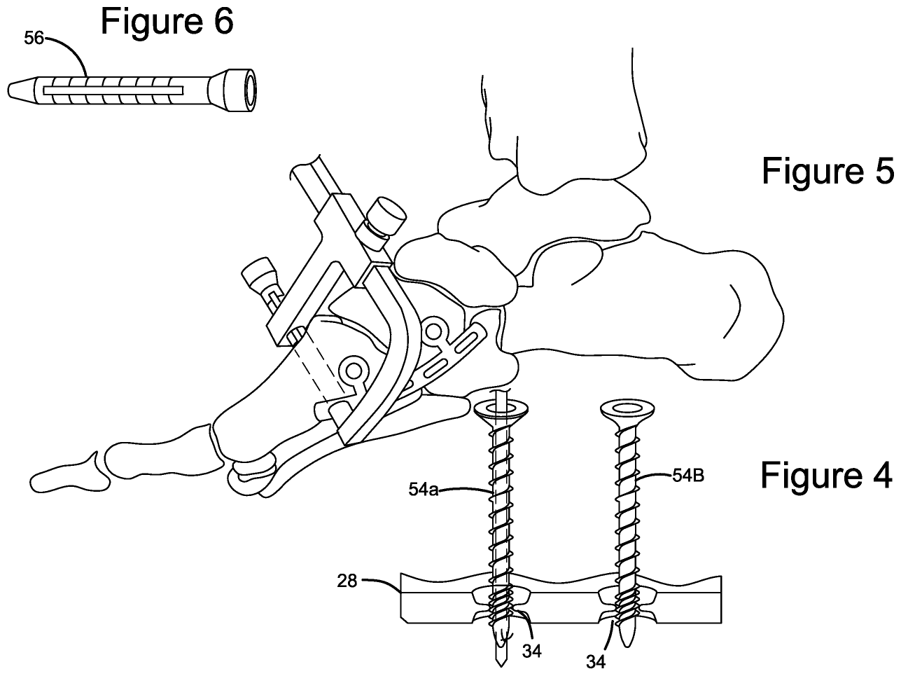

FIG. 4 shows two different embodiments of reverse locking screws that can be used to place locking plate 28 of the present invention. Reverse locking screw 54a is a cannulated screw type whereas reverse locking screw 54b is a solid screw type. Both reverse locking screws 54a or 54b work such that the screws become fixed into locking plate 28 at a distal portion of the screw, as opposed to the proximal end of most normal screw types. As will be discussed in more detail below, the reverse locking screws 54a or 54b of the present invention work by being placed from a dorsal position of the foot when locking plate 28 is placed from a plantar position of the foot. Being able to position the reverse locking screws 54a or 54b from a dorsal position allows for a medial dorsal midfoot incision over the first metatarsocuneiform joint 16, which is far more precise and less damaging to the foot due to less invasive dissection than positioning a screw from a plantar position.

FIG. 5 shows a foot having a reverse locking plate 28 of the present invention being placed. Prior to the view shown in FIG. 5, the first step of placing reverse locking plate 28 of the present invention is joint preparation and correction alignment. Then, locking plate 28 is temporarily fixated to the joint at the medial aspect of the foot using temporary normal fixation screws/pins or temporary plate tacks (not shown) within the two fixation apertures 32. By the term normal screws, it is meant that the screws are not of the reverse locking type. Next, locking guide 36 is placed plantarly by having locking pins 52 lock into place with the appropriate locking pin holes 35 such that aperture 50 of locking piece 48 is aligned with a locking aperture 34. Once the locking guide 36 has been placed, a cannulated measuring device 56, such as shown in FIG. 6, is placed within aperture 46 of upper adjustable arm 42 of locking guide 36, such as shown in FIG. 5. Cannulated measuring device 56 allows for accurate screw length and becomes externally locked to maintain accurate orientation of the drill. Then, a pilot hole for a reverse locking screw 54a or 54b is drilled in a cannulated manner while locking guide 36 is held in place plantarly.

Once a pilot hole has been drilled, a reverse locking screw 54 is then placed using standard technique. As stated above, reverse locking screw 54a or 54b will become locked once it contacts the plate. As shown in FIG. 4, in some embodiments, reverse locking screw 54a or 54b will travel slightly past the depth of a locking aperture 34 of locking plate 28 before it becomes locked in place. In one or more embodiments, only four reverse locking screws 54 are placed, two on each side of the first metatarsocuneiform joint 16, although more reverse locking screws 54 can be placed as needed. Once all of the reverse locking screws 54 have been placed in the locking apertures 34 of locking plate 28, the temporary fixation screws placed in the fixation apertures 32 can be removed and additional more permanent standard screws can be placed within fixation apertures 32.

The method of using locking guide 38 to place locking plate 28 using reverse locking screws 54a or 54b creates a stronger locked plantar construction that will allow for early mobilization and weight bearing by the patient. This method and locking plate technology may be applied to all joints of the foot where fixation strength is needed.

In light of the foregoing, it should be appreciated that the present invention significantly advances the art by providing a plantarly applied reverse locking plate and method of use that is structurally and functionally improved in a number of ways. While particular embodiments of the invention have been disclosed in detail herein, it should be appreciated that the invention is not limited thereto or thereby inasmuch as variations on the invention herein will be readily appreciated by those of skilled in the art. The scope of the invention shall be appreciated from the claims that follow.

What is claimed is:

1. A method of placing a plate fixation device in a foot comprising the steps of:
a. plantarly placing a reverse locking plate on the foot; and
b. utilizing a locking guide to dorsally place reverse locking screws into the foot to mate with the reverse locking plate.

2. The method of claim 1, wherein the reverse locking plate includes a plurality of fixation apertures, a plurality of locking apertures, and a plurality of locking pin-holes located adjacent each of the plurality of locking apertures.

3. The method of claim 2, wherein the locking guide includes a locking piece comprising an aperture and two locking pins.

4. The method of claim 1, further comprising the step of temporarily fixating the locking plate at a medial aspect of the foot by utilizing the plurality of fixation apertures.

5. The method of claim 4, further comprising the step of plantarly placing the locking guide by having the locking pins lock into place with locking pin-holes such that the locking guide aperture is aligned with one of the plurality of locking apertures of the reverse locking plate.

6. A reverse locking plate for use in foot surgery comprising:
a. a body having an upper and lower surface with a side portion located between said upper and lower surface;
b. a plurality of fixation apertures protruding from the side portion to lie above said upper surface;
c. a plurality of locking apertures running from the upper to lower surface of the body; and
d. a plurality of locking pin-holes located adjacent each of the plurality of locking apertures on the lower surface of the body.

7. The reverse locking plate of claim 6, wherein the reverse locking plate is made from a metal selected from the group consisting of medical grade surgical steel or titanium.

8. A kit for use in foot surgery, the kit comprising
a. a reverse locking plate; and
b. a locking guide.

9. The kit of claim 8, wherein the reverse locking plate comprises:
a. a body having an upper and lower surface with a side portion located between said upper and lower surface;
b. a plurality of fixation apertures protruding from the side portion to lie above said upper surface;
c. a plurality of locking apertures running from the upper to lower surface of the body; and
d. a plurality of locking pin-holes located adjacent each of the plurality of locking apertures on the lower surface of the body.

10. The kit of claim 9, wherein the locking guide comprises:
a. a vertical arm;
b. a lower set arm; and
c. an upper adjustable arm.

11. The kit of claim 10, wherein the lower set arm includes a locking piece at a distal end of the lower set arm wherein the locking piece includes an aperture and two locking pins.

12. The kit of claim 11, wherein the upper adjustable arm is adjustable along the vertical arm and is adjusted by use of an adjustment means and wherein the upper adjustable arm includes an aperture at a distal end of the upper adjustable arm.
